(19) United States
(12) Patent Application Publication
(10) Pub. No.: US 2015/0044136 A1
(43) Pub. Date: Feb. 12, 2015

(54) Ophthalmological treatment for michobial infections

(71) Applicants: George H. Scherr, Highland Park, IL (US); Seth I. Kaplan, Chicago, IL (US)

(72) Inventors: George H. Scherr, Highland Park, IL (US); Seth I. Kaplan, Chicago, IL (US)

(21) Appl. No.: 13/987,592

(22) Filed: Aug. 12, 2013

(51) Int. Cl. A61K 47/26; A61K 31/7004; A61K 51/06; A61K 39/395

(52) U.S. Cl. CPC A61K 47/26; A61K 39/395; A61K 31/7004; A61K 51/06
USPC 424/1.73; 514/777; 424/130.1; 514/23; 536/2

(57) ABSTRACT

The invention described herein relates to the composition of a liquid in which a silver ion moiety can act as a preservative as well as an astringent for the inhibition of microbial infections of the eye.

### DESCRIPTION OF THE PRIOR ART

The development of calcium alginate fibers by Courtaulds in Coventry, England, principally for the textile industry, prompted the investigation by researchers such as Scherr to prepare medical dressings from the calcium alginate fibers, especially in the light of their hemostatic activity and slight antimicrobial activity. (See U.S. Pat. No. 5,674,524 issued Oct. 7, 1997, entitled Alginate Fibrous Dressings and Method of Making; U.S. Pat. No. 5,718,916 issued Feb. 17, 1998, entitled Alginate Foam Products; U.S. Pat. No. 7,128,929 issued Oct. 31, 2006, entitled Alginate Foam Products.)

The increased interest in calcium alginate fiber dressings for the treatment of wounds further resulted in the incorporation of antimicrobial agents in such dressings. The resultant development by Scherr of a silver alginate moiety introduced antimicrobial activity for medical dressings that had a number of advantages over antibiotics. (See U.S. Pat. No. 6,696,077 issued Feb. 24, 2004, entitled Silver Alginate Foam Compositions, India Patent Number 221859, issued Jul. 8, 2008, entitled Alginate Foam Compositions; United Kingdom Patent number GB 2,357,765, issued Apr. 21, 2004, entitled alginate Foam Compositions; Russian Patent Number 2322266, issued Feb. 18, 2003, entitled Alginate Foam Compositions).

Aqueous insoluble salts of alginate are readily achieved with salts of calcium, aluminum, zinc, copper, iron, and silver, in which case, the insoluble alginate salt is suspended in an aqueous gel. A detailed discussion of the properties of alginates and the preparation of their solid states has been published by McDowell in his book, Properties of Alginates.

Pectins, either alone or in conjunction with alginates have also been used in the preparation of products that are amenable to the manufacture of wound dressings. Pectins are polysaccharides that contain a 1,4-linked α-D-galactosyluronic acid residue. There are essentially three pectic polysaccharides that are isolated from primary cell walls and they can be characterized as:

1. Homogalacturonans

2. Substituted galacturonans

3. Rhamnogalacturonans

Pectins may be esterified with methanol and the pectins can be classified as high- or low- ester pectins. The low-ester pectins usually require calcium to form a gel. A detailed presentation of the chemistry of pectins has been published by the American Chemical Society and edited by Marshall L. Fishman and Joseph J. Jen (Chemistry and Function of Pectins, 1986).

Christian Bannert describes the preparation of a gel that can be utilized as a dressing into which gel disinfectants and medications can be added for treating the mucosa. These gels are obtained by using aqueous soluble alginates which can react with a calcium salt. Calcium salts can react with a pectin which has a low degree of esterification. (See U.S. Pat. No. 5,147,648, issued Sep. 15, 1992).

U.S. Pat. No. 5,688,923 by Gerrish issued Nov. 18, 1997, describes a process of making pectin fibers that can be utilized in wound dressings.

U.S. Pat. No. 3,639,575 issued Feb. 1972 to Irving R. Schmolka is concerned with the treatment of burns utilizing water soluble silver salts and a matrix for those silver salts which are composed of aqueous gels of polyoxyethylene polyoxypropylene block copolymers. All seven claims of the Schmolka patent require that the composition prepared is designed “. . . to treat a burn wound.” A silver water soluble salt may be used which can be silver nitrate, silver sulfate, silver acetate, and/or silver lactate monohydrate; these silver salts must dissolve in water at a minimum concentration 0.1 per cent.

The gels as prepared in the examples cited in U.S. Pat. No. 3,639,575 are transparent and form a clear gel at room temperature. When the gels are cooled, ostensibly below the temperature of room temperature, they become liquids.

Studies examining the mechanism of action of silver in demonstrating antimicrobial activity led various workers to conclude that the presence of silver ions was more important than the amount of silver metal as colloidal silver or the time that the substrate containing microbes was exposed to the silver. Silver was prepared in the form of a spongy metallic form or by coating various products which contain large surfaces such as sand by K. Süpfle and R. Werner (1951 Mircrodeosimetric investigation of the oligodynamic effect of silver. Mikrochemie ver. Mikrochim. Acta., 36/37, 866-881). These workers showed that E. coli placed in flasks having counts of 18,000 E. coli per ml of water, when such counts were exposed to flasks containing sand coated with silver at a concentration of 10 per cent silver of the amount of sand, would result in a sterile environment in four hours. Even counts as high as 120,000 E. coli per ml of solution, resulted in sterility in 24 hours. The relatively low count of E. coli of 1.8×10³ and the very high concentration of silver coated onto the sand of 10 per cent would readily explain a sterile environment in four hours. However, in vivo experiments reports in 510(k) reports that are available have never utilized a silver concentration as high as 10 per cent which could be toxic to a patient and result in argyria.

Azary Technologies LLC has produced a silver-containing fabric in which a nylon-containing textile fiber which contains silver which has been plated onto the nylon. The Azary company in submitting data pertinent to their United States Food and Drug Administration 510(k) device, K040518, has indicated that the silver contained on the nylon fiber is pure elementary silver at a purity of 99.9 per cent. These textiles coated with elemental silver have been approved for coating nylon which can be used in a medical dressing or in certain wearing apparel such as socks. Here too, it is expected that the elemental silver will have to be converted to ionic silver by some mechanism in order to ensure antimicrobial activity.

The biocidal composition of silver ions and sodium polypectate described in James W. Van Leuven's U.S. Pat. No. 4,184,974 is used in the range of 100 to 400 parts per million, also includes in claim 1, a silver ion in the range from about 13 to 250 parts per million, and glycerin in the range of about 4 to 10 per cent by weight; in addition, the pH of the water soluble base should be in the range of 7.2 to 7.8. The inventor submits that the sodium polypectate may be prepared by treating pectin with sodium carbonate in order to solubilize the pectins. The inventor therefore characterizes pectin as a water insoluble compound which is inconsistent with data submitted by innumerable documents that describe the utilization of pectins. The American chemical Society monograph, Chemistry and Function of Pectins, unequivocally states that “Pectins are soluble in pure water.” The only caveat the ACS monograph submits is the case where “. . . they (pectins) are insoluble in aqueous solutions in which they would gel at the same temperature if dissolved at a higher temperature.” However, that characterization for pectins when they become insoluble is totally irrelevant to the specification and claims of U.S. Pat. No. 4,184,974. The inventor of this patent also submits that the preparation of sodium polypectate in order to solubilize the pectin with sodium carbonate also serves to result in the polypectate chelating readily with the alkaline earth cations such as calcium and magnesium. The reaction of calcium with pectin is well known and has been described in innumerable documents. The American Chemical Society's Symposioum on pectins cited above also characterizes on page 8 that pectin will gel in the presence of divalent cations and increasing the concentration of divalent cations, such as calcium, increases the gelling temperature and gel strength.

In the patent by Gerrish, et al. (U.S. Pat. No. 5,688,923 issued Nov. 18, 1997), the inventors show that pectin can readily be dissolved in water as is indicated for example, in their claim 29. Their claim 35 indicates pectins will react with polyvalent cations wherein the cation which may consist of calcium, copper, barium, magnesium, zinc, and iron and will precipitate as an insoluble fiber of a pectate. The inventors have made no claim nor conducted any experiments as described in their U.S. Pat. No. 5,688,923 concerning the ability of silver ions to precipitate pectins. Also, they claim in their claim 6 that their claim 1 indicates they have non-pectin polysaccharides added to it such as hyaluronic acid, carrageenan, alginic acid, or sodium alginate. Alginic acid is insoluble in water, as has been well described in the chemical literature, but sodium alginate when added to the pectin, where a fiber is to be made, will certainly enhance the strength of the fiber.

Consequently, it is not clear and inconsistent with known chemistry why U.S. Pat. No. 4,184,974 of Van Leuven finds it necessary to solubilize pectins, which are known to be soluble in water, and that the preparation of a sodium polypectate is deemed necessary in order for the pectin to chelate with alkaline earth cations such as calcium when pectins readily chelate with calcium and become insoluble as has been well described in the literature.

The seventh edition of The Merck Index indicates that pectin is “completely soluble in 20 parts of water forming a viscous solution containing negatively charged hydrated particles.” For use as a medical product, pectin per se, as a powder was recommended in The Merck Index for hemostatic effect and as a paste for treatment of decubitus ulcers.

The MSDS sheet of Spectrum Laboratory Products, Inc., Gardena, Calif., 90248 dated Sep. 13, 2006 essentially paraphrases the data for pectin that The Merck Index cited above as being soluble in 20 parts of water and dissolves more readily in water if it is first moistened with alcohol or glycerol. The synonyms for pectin manufactured by Spectrum Laboratory Products, Inc. indicates that it maybe also cited as Methoxypectin, Methyl pectin, Methyl pectinate, Pectinate, Pectrinic acid, Pectins, and Colyer Pectin. Chapter 5, in the book by A. Nussinovitch provides an excellent review of the chemistry of pectins, their preparation, and their reactions.

A singular aspect of the chemical activity of silver and silver salts has to do with the ease with which silver ions combine with proteins, oxygen, and various halogens to result in insoluble material. Thus for example, oxides of silver (as Ag₂O or Ag 0) are considered insoluble in water as is silver chloride. L. Goodman and A. Gilman (The Pharmacological Basis of Therapeutics, 3^{rd} Ed. New York, The Macmillan Company 1965) reported that the toxic effects of silver compounds on microorganisms are probably due to the silver ions which precipitate the protein of bacterial protoplasm. It is very well known that soluble silver salts such as silver nitrate will quickly precipitate protein and oxidize to a dark brown or black precipitate. The silver protein complex so formed contributes to a sustained antimicrobial action by slowly liberating small amounts of silver ions. It therefore would be necessary for the silver metal, in the report by Illingworth, et al. for the device in repairing a diseased or damaged heart valve, to be ionized to the silver ionic state.

Neither silver ions nor silver colloids should be released into a wound in a relatively large amount in a short period of time; otherwise the patient will turn blue as a result of silver poisoning (argyria) Trop et al., 2006).

### BACKGROUND OF THE INVENTION

The 18^{th} revision of the United States Pharmacopeia which provides the official data from Sep. 1, 1970, describes the preparation of a silver nitrate ophthalmic solution on page 602 as a topically anti-infective to be applied to the conjunctiva, it stipulates the use of a 0.1 ml of a 1% solution. Although millions of newborn babies had been treated with the silver nitrate solution in order to protect them against microbial infections that they may acquire during the process of delivery (ophthalmia neonatorum), the use of silver nitrate has been discontinued in most countries of the world due to the strong chemical irritation which frequently results from the use of silver nitrate on human tissues and especially in the eye. Consequently, most countries adopted the use of neomycin or chloramphenicol eye drops as a substitute for silver nitrate.

With the advent of the reliance on antibiotics for the treatment of ocular infections, the antibiotics that were available for such treatment included erythromycin, polymyxin B, tobramycin, gentamicin, a number of fluoroquinolones, and others. The emergence of bacterial resistance to antibiotics began to limit the effectiveness of many of the antibiotics that were relied on. It became necessary to alter antibiotics when resistance was apparent. It also delayed treatment until a different antibiotic that was effective was selected and delayed the resolution of the infection in many cases.

The detailed report by Meaders and Azar (2012) describes in details the marked increase in antibiotic resistance resulting from their substitution of the silver nitrate. This further resulted in loss of work to patients who required a longer period for therapy and a marked increase in cost of treatment.

Despite the fact that silver nitrate has been discontinued for the treatment of ophthalmia neonatorum, there are still companies promoting argyrol preparations for the treatment of preventative infections of the eye. The company, Argyrol Pharmaceuticals Corporation, promotes and sells a 10% silver protein preparation labeled Argyrol®. It is not clear from the literature provided whether Argyrol has all of the silver covalently linked to a protein, nor in fact what the protein is or whether some of the silver is free in the colloidal or in the ionic state as a salt.

Literature of the Argyrol Pharmaceuticals Corporation recommends the use of their 10% silver protein argyrol preparation to be placed in the eye:

“In the EYE: use as an aid in cleansing eye of germs and/or particles, mucous and debris producing irritation or discomfort.

Drop 1 to 3 drops into each eye. Repeat if necessary in 4 hours.”

The directions also recommend that the product not be used for more than 72 hours, but it is not clear whether this includes or excludes its use for the treatment of infected eyes.

The Alcon Company promotes a prescription product manufactured by the Cardinal Health Company in Woodstock, Ill., which is a Betadine® (povidone-iodine) solution. The Betadine solution is characterized as a “povidone-iodine” which is a broad-spectrum microbicide with the chemical formulas: 2-pyrrolidinone, 1-ethenyl-, homopolymer, compound with iodine; 1-vinyl-2-pyrrolidinone polymer, compound with iodine. the clinical pharmacology cited for this product indicated:

“A placebo-controlled study in 38 normal volunteers yielded data for 36 subjects who showed a mean log₁₀ reduction of 3.05 log₁₀ units for aerobes in 10 minutes following prepping the skin with Betadine 5% Sterile Ophthalmic Prep Solution compared with reduction of 1.58 log₁₀ units after prepping with vehicle free of the iodine complex.”

The directions for utilizing the Betadine solution in treating an ophthalmic infection is cited as:

“While separating the lids, irrigate the cornea, conjunctiva, and palpebral fornices with Betadine 5% Sterile Ophthalmic Prep Solution using a sterile bulb syringe. After the Betadine 5% Sterile Ophthalmic Prep solution has been left in contact for two minutes, sterile saline solution in a bulb syringe should be used to flush the residual prep solution from the cornea, conjunctiva, and the palpebral fornices.”

Although the Betadine solution is reported in achieving a reduction of 3.05 log₁₀ units for aerobes in 10 minutes, the directions for using the product clinically indicates that it should be immediately washed from the site after exposure of only 2 minutes in order to flush out the residual solution from the cornea, conjunctiva, and the palpebral fornices.

There is no other data supplied in this report to indicate what organism was used or why they accepted reduction of the log of the organisms used of 3.05 units for an aerobic organism in 10 minutes requires that the solution be washed from the cornea and conjunctiva after being left in contact for only two minutes.

The product Moxeza® which is a 0.5% moxifloxacin HCl ophthalmic solution, is an antibiotic eye drop used to treat the most common eye infection, bacterial conjunctivitis. This product is licensed to the Alcon company by Bayer Schering Pharma AG.

The directions for use of the Moxeza® product indicate that it is to be “. . . dosed two times per day for seven days and is indicated for patients four months of age and older.”

The Alcon company also prepares a product called Vigamox® which is a 0.5% moxifloxacin HCl ophthalmic base, also used as an antibiotic eye drop in treating the most common eye infections and bacterial conjunctivitis. The Vigamox® solution ostensibly of the same concentration of moxifloxacin as the Moxeza® solution is recommended to be “dosed three times per day for seven days and is indicated for patients one year and older.” The safety and efficacy data for Vigamox® is indicated as occurring with decreased visual acuity, and also possible occurrence of burning or stinging, and red or itchy eyes. The company indicates that the patient using this product may suffer some “other effects” but they do not indicate specifically what these other effects may be.

### SUMMARY OF THE INVENTION

### Antimicrobial Experiment I

### Bacterial Culture Preparation

Klebsiella pneumoniae (ATCC BAA 1705), S. aureus (MRSA) ATCC 700609, Staphylococcus epidermidis (ATCC 155) Acinetobacter baumannii (ATCC 19606), Proteus hauseri (ATCC 13315), Serratia marcescens (ATCC 13880), Enterobacter aerogenes (ATCC 13048). Pseudomonas aeruginosa (ATCC 15442) and Staphylococcus aureus (ATCC 6538) stock cultures were obtained from American Type Culture Collection (ATCC, VA) or Microbiologics Inc. (MN) and maintained as per suppliers' recommendations.

For challenge experiments, a single colony from the plate stock was transferred to 10 ml of Tryptic Soy Broth (TSB, Neogen), or Brain Heart Infusion Broth (BHI, Neogen), and incubated at 36.5° C. for 16-20 hours. An aliquot of the culture was then transferred to a fresh TSB or BHI tube and grown for an additional 16-20 hours at 36.5° C. prior to the day of the challenge study. On the day of the challenge study, the broth culture was mixed at high speed (Vortex Mixer) until homogenous. This suspension was used for the challenge studies described.

A silver alginate composition was prepared using the following ingredients and encoded 127-35B.

| | | |
|---|---|---|
| Sodium alginate | 20 | gms |
| DI water | 375 | ml |
| Cellulose (high density) | 0.2 | gms |
| White petrolatum | 4.6 | gms |
| White mineral oil | 11.0 | ml |
| Sodium lauryl sulphate | 1.5 | gms |
| Aerosol OT | 3.0 | ml |
| Silver Nitrate | 1.0 | gms |

### Challenge Study

The provided suspension 127-35B was vortexed until homogeneous. For each of the bacterial species challenge, a duplicate one gram aliquot of each formulation was added to one of each of 100 ml DI water in a separate sterile Erlenmeyer flask. One hundred ml of the bacteria suspension was added to the test product and a NIST traceable laboratory timer was started. The mixture of the test product and bacterial solution was homogenized (by stir plate) for 30, 60, 90, 120 and 150 minutes. After each specified time interval (30, 60, 90, 120, or 150 minutes) an aliquot of the solution was removed and immediately combined with an equal amount of 2× D/E Neutralizing Broth (Enzymatic Digest of Casein, Yeast Extract, Dextrose, Sodium Thioglycollate, Sodium Thiosulfate, Sodium Bisulfite, Lecithin, Bromcresol Purple, Polysorbate 80; Accumedia, MI) to neutralize the antimicrobial properties of the test article. Duplicate samples, at each time point, were collected and neutralized. Dilutions of 1/100 were conducted in phosphate buffered water (3M, USA) to further reduce any residual antimicrobial properties. Aliquots from each neutralized challenge suspension were plated directly in duplicates of 100 μand 1000 μl onto nutrient non-selective agar plates (TSA or TSA II).

The plates were placed into 36.5°±1° C. incubator for 24±2 hours. Following incubation time the plates were removed from the incubator and the colony-forming units (CFU's) were enumerated and recorded. Any suspect plates were placed back in the incubator for an additional 24 hours to monitor for additional growth. Positive, negative, and neutralization controls were performed along with the test subjects to provide quality control and reference data.

The results of the above study are summarized in the following tables one (1) and two (2). The results presented pertain only to the study conducted on the test articles. The results presented pertain only to the samples analyzed and identifier number(s) indicated. Neutralization controls were performed as outlined in the method and as per Good Laboratory Practices. All analyses were performed in accordance to laboratory practices and procedures set-forth by our NELAP/TNI accreditation standards (ISO 17025).

TABLE 1
The recovery of indicated bacterial species at the various time points following exposure to the provided Silver Preparation 127-35B

| | 0 | 30 Minutes | | 60 Minutes | | 90 Minutes | | 120 Minutes | | 150 Minutes | |
|---|---|---|---|---|---|---|---|---|---|---|---|
| Micro- | seconds/ | CFU/ | Percent | CFU/ | Percent | CFU/ | Percent | CFU/ | Percent | CFU/ | Percent |
| organism | Initial * | ml | Reduction | ml | Reduction | ml | Reduction | ml | Reduction | ml | Reduction |
| S. epidermis | 9.2 × 10⁵ | <0.5 | >99.99994% | <0.5 | >99.99994% | <0.5 | >99.99994% | <0.5 | >99.99994% | <0.5 | >99.99994% |
| ATCC#155 | | | | | | | | | | | |
| P. auruginosa | 1.6 × 10⁶ | 228 | 99.99% | <0.5 | >99.99996% | <0.5 | >99.99996% | <0.5 | >99.99996% | <0.5 | >99.99996% |
| ATCC# 15442 | | | | | | | | | | | |
| A. baumanni | 1.2 × 10⁶ | <0.5 | >99.99995% | <0.5 | >99.99995% | <0.5 | >99.99995% | <0.5 | >99.99995% | <0.5 | >99.99995% |
| ATCC# 19606 | | | | | | | | | | | |
| P. hauseri | 9.6 × 10⁵ | 30 | 99.997% | 33 | 99.997% | <0.5 | >99.99994% | <0.5 | >99.99994% | <0.5 | >99.99994% |
| ATCC# 13315 | | | | | | | | | | | |
| S. marcescens | 1.4 × 10⁶ | <0.5 | >99.99996% | <0.5 | >99.99996% | <0.5 | >99.99996% | <0.5 | >99.99996% | <0.5 | >99.99996% |
| ATCC# 13880 | | | | | | | | | | | |
| S. aureus | 9.4 × 10⁶ | 563 | 99.994% | 278 | 99.997% | 1590 | 99.983% | 858 | 99.991% | 28 | 99.9997% |
| (MRSA) | | | | | | | | | | | |
| ATTC#700699 | | | | | | | | | | | |
| K. pneumonia | 1.1 × 10⁶ | <0.5 | >99.99995% | <0.5 | >99.99995% | <0.5 | >99.99995% | <0.5 | >99.99995% | <0.5 | >99.99995% |
| ATCC# | | | | | | | | | | | |
| BAA1705 | | | | | | | | | | | |
| E. aerogeous | 1.7 × 10⁶ | 49 | 99.997% | <0.5 | >99.99997% | <0.5 | >99.99997% | <0.5 | >99.9997% | <0.5 | >99.99997% |
| ATCC# 13048 | | | | | | | | | | | |
| S. aureus | 2.5 × 10⁶ | 5318 | 99.8% | 84 | 99.997% | 0.5 | 99.99998% | 4 | 99.9998% | 0.5 | 99.99998% |
| ATCC# 6538 | | | | | | | | | | | |

* Initials were calculated based on the average of PBS controls (20 ml PBS spiked with 0.1 ml bacterial solution) and Neutralization Buffer controls (20 ml neutralizing buffer spiked with 0.1 ml of the bacterial solution). PBS and Neutralizing Buffer controls were diluted in Phosphate Buffer Solutions and plated in 0.1 and 1.0 ml duplicates. Neutralization controls (2.0 grams soap mixed with 18 ml neutralization buffer And spiked with 0.1 ml bacterial solution) were performed for each microorganism and plated in 0.1 ml and 1.0 ml at a 1/1000 dilution. Recoveries from neutralization controls, PBS controls, and neutralizing buffer controls did not vary significantly in the concentration of the added bacterial solution/species except where indicated.
** The calculation of each percent reduction time point is based upon results from 1.0 volumes at a 1/100 dilution performed in duplicate.

### Cytotoxicity Evaluation of the Supplied Liquid Sample 127-35B.

### ASTM F895-84 Test Method was Utilized.

The Sample was analyzed as described in ASTM F895-84 and ISO 10993-5. Monolayers of L929 cells were grown in six 150 mm cell culture plates (Corning, USA). Following 24 hours to cell passage, the plates were over-layed with agar supplemented dMEM/FBS media as described by the ASTM method. One hundred milligrams of the provided samples was added to the center of four of the over-layed wells. One well was left undisturbed as this served as negative control. An additional well was inoculated with a 2 cm² piece of latex rubber. The cells were then incubated in 5% carbon dioxide at 36.5° C.±1 for 24 hours. The plates and cells were evaluated macro and microscopically for signs of malformation, degeneration, sloughing, or lysis of the cells within the zone directly beneath and surrounding the discs. Cells were then re-incubated for an additional 24 hours at the conditions described above and evaluated again. The size of the zone surrounding the inoculated area was evaluated for signs of cell growth inhibition and/cell lysis.

The numerical evaluation is presented in the following tables. Table 3 contains the numerical values describing the size of zone of lysis surrounding the inoculums as per the ASTM standards. Table 4 contains the relative numerical value describing the number of cells affected within the zone of inhibition or toxicity. Each sample observed is assigned a numerical value based on cellular degradation and/or cell death. The interpretation of the assigned numerical values is presented in Table 5 and Table 6.

Briefly, number 0=unchanged/no evident cytotoxicity; 5=complete monolayer destruction and severe cytotoxicity. The ASTM and ISO 10993-5 consider a scoring of 3 or higher as toxic.

TABLE 3
Description of the size of zone of lysis surrounding
the test sample as per ASTM standards.

| | Zone Description Following | | | | Zone Description Following | | | |
|---|---|---|---|---|---|---|---|---|
| SAMPLE | 24 Hour Incubation* | | | | 48 Hour Incubation* | | | |
| 127-35B | 1 | 1 | 1 | 0 | 2 | 2 | 2 | 1 |

| | | |
|---|---|---|
| (+) Control | 4 | 5 |
| (% Phenol) | | |
| (−) CONTROL | 0 | 0 |

*0 = unchanged; 5 = complete monolayer destruction and severe cytotoxicity.

TABLE 4
Description of the condition of cells within the zone of lysis
surrounding the test sample as per the ASTM standards

| | Cell Description Following | | | | Cell Description Following | | | |
|---|---|---|---|---|---|---|---|---|
| Sample | 24 Hour Incubation* | | | | 48 Hour Incubation* | | | |
| 127-35B | 1 | 1 | 1 | 1 | 1 | 2 | 1 | 1 |

| | | |
|---|---|---|
| (+) Control | 5 | 5 |
| (0.5% Phenol) | | |
| (−) CONTROL | 0 | 0 |

*0 = unchanged; 5 = complete monolayer destruction and severe cytotoxicity

TABLE 5
Zone Description (for data presented in Table 3)

| Zone Index | Description of Zone |
|---|---|
| 0 | No detectable zone around or under specimen |
| 1 | Zone limited to area under specimen |
| 2 | Zone extends less than 0.5 cm beyond specimen |
| 3 | Zone extends 0.5 to 1.0 cm beyond specimen |
| 4 | Zone extends greater than 1.0 cm beyond specimen but |
| | does not involve entire dish |
| 5 | Zone involves entire dish |

TABLE 6
Lysis Description (for data presented in Table 4)

| Zone Index | Description of Zone |
|---|---|
| 0 | No observable cytotoxicity |
| 1 | Less than 20% of zone affected |
| 2 | 20-39% of zone affected |
| 3 | 40 to 59% of zone affected |
| 4 | 60 to 80% of zone affected |
| 5 | Greater than 80% of zone affected of zone affected |

### DESCRIPTION OF THE PREFERRED EMBODIMENTS

Although the preparation 127-35B showed an acceptable lack of tissue toxicity in accord with criteria established by ASTM F895-84 and ISO 10993-5, a drop of 127-35B was instilled into the eye of a human male volunteer with the result that the eye developed a moderate amount of erythema and the patient complained about a more than moderate amount of burning feeling in the eye. Consequently, the formulation was modified as indicated by the code number 127-39T.

### Composition of 127-39T

| | |
|---|---|
| H₂O deionized or distilled | 228 ml |
| Pectin | 3.25 gm |

The water should be heated to boiling, if necessary, to dissolve the pectin completely. Use a Pyrex flask with a magnetic stirrer running continuously.

| | | |
|---|---|---|
| Na alginate | 5.0 | gm |
| White mineral oil | 20.0 | ml |
| White petrolatum | 6.5 | gm |
| Cellulose, crystalline | 1.0 | gm |
| Aerosol OT | 2.0 | ml |
| AgNo3 | 1.0 | gm |
| Lactose | 0.5 | gm |
| Dextrose | 1.0 | gm |

All components must be added with continuous stirring

A drop of the preparation 127-39T was instilled into the eye of the same male human volunteer and the result was acceptable in that erythema was very slight or minimal and no burning sensation was experienced. Accordingly, the next experiment assessed the effect of the new silver preparation, 127-39T, on three organisms and concomitantly assessed the effect of two antibiotics on the same organisms.

The Tobrex 0.3% ophthalmic ointment was procured by prescription. The ointment is manufactured by the Alcon Company and is contained in a small tube suitable for ophthalmic treatment; the total amount being 3.5 gm. The inactive components for the tobramycin ophthalmic ointment are the white mineral oil and white petrolatum.

The Ciloxan 0.3% ophthalmic ointment is also manufactured by the Alcon Company; the active ingredient being ciprofloxacin hydrochloride in an amount of 3.33 mg equivalent to a 3 mg base. The inactive ingredients are mineral oil and white petrolatum.

The preparation 127-39T as well as the Ciloxan and the Tobrex ophthalmic ointments were stored at room temperature until use in the challenge studies.

### Bacterial Culture Preparation

Klebsiella pneumoniae (NDM-1 strain ATCC BAA 1705), Pseudomonas aeruginosa (ATCC 15442), and Vancomycin Resistant Entercoccus facium (VRE, ATCC 700221) stock cultures were obtained from American Type Culture Collection (ATCC, VA) or Microbiologics Inc. (MN) and maintained as per suppliers recommendations. For challenge experiments, a single colony from the pure plate stock was transferred to 10 ml of Tryptic Soy Broth (TSB, Neogen), and incubated at 36.5° C. for 16-20 hours. An aliquot of the culture was then transferred to a fresh TSB tube and grown for an additional 16-20 hours at 36.5° C. prior to the day of the challenge study. On the day of the challenge study, the broth culture was mixed at high speed (Vortex Mixer) until homogenous. This suspension was used for the challenge studies described.

### Challenge Study: Nov. 14, 2012 to Nov. 19, 2012

The provided formulations were vortexed until homogeneous if necessary. For each of the requested bacterial species challenges, one gram aliquot of each formulation was added to each of two separate sterile Erlenmeyer flasks containing 100 mL of reagent grade laboratory water (Class I ASTM). The aqueous suspension was mixed vigorously for 20 minutes to ensure the homogenous dispersion of the suspension.

The temperature of the test articles and their suspension prior to and during efficacy testing was maintained at 21-23° C. One hundred milliliters of the previously described homogenous bacteria suspension was added to each of the flasks containing the test article suspension and a NIST traceable laboratory timer was started. The mixture of the test article suspension was homogenized (by stir plate) for 30, 60, 90, 120 and 150 minutes. After each specified time interval (30, 60, 90, 120, or 150 minutes) an aliquot of the test article solution was removed and immediately combined with an equal amount of 2× D/E Neutralizing Broth (Enzymatic Digest of Casein, Yeast Extract, Dextrose, Sodium Thioglycollate, Sodium Thiosulfate, Sodium Bisulfite, Lecithin, Bromcresol Purple, Polysorbate 80; Accumedia, MI) to neutralize the antimicrobial properties of the test article. Duplicate samples, at each time point, were collected and neutralized. Dilutions of 1/100 were conducted in phosphate buffered water (3M, USA) to further reduce any residual antimicrobial properties. Aliquots from each neutralized challenge suspension were plated directly and at 1/100 dilution in duplicates of 100 μand 1000 μl onto nutrient non-selective agar plates (TSA). Positive control flasks containing reagent grade water and inoculated with the respective bacterial species served as positive controls. The positive control flasks were treated similarly to the challenge study flasks and were samples 0, 90, and 150 minutes just like the challenge flasks. The collected neutralized samples from the control flasks were diluted 10⁻³ in phosphate buffered dilution water and plated onto TSA.

The TSA Plates were placed into 36.5°±1° C. incubator for 22±2 hours. Following incubation time the plates were removed from the incubator and the colony forming units (CFU's) were enumerated and recorded. Any suspect plates were placed back in the incubator for an additional 24 hours to monitor for additional growth. Recovery from positive control plates did not vary throughout the term of the study indicating minimal loss of viability due to osmotic stress. Average recoveries from the positive control plates served as initials for calculating the average reduction efficacy of each suspension at each time point.

The results of the above study are summarized in the following Tables 7, 8, and 9.

Positive, negative, and neutralization controls were performed as outlined in the method and as per Good Laboratory Practices. All analyses were performed in accordance to laboratory practices and procedures set-forth by our NELAP/TNI accreditation standards (ISO 17025).

TABLE 7
The recovery of indicated bacterial species at the indicated time points following exposure to the preparation 127-39T

| | | Exposure Time | | | | | | | | | |
|---|---|---|---|---|---|---|---|---|---|---|---|
| | | 30 Minutes | | 60 Minutes | | 90 Minutes | | 120 Minutes | | 150 Minutes | |
| Micro- | | CFU/ | Percent** | CFU/ | Percent** | CFU/ | Percent** | CFU/ | Percent** | CFU/ | Percent** |
| organism | Initial* | ml | Reduction | ml | Reduction | ml | Reduction | ml | Reduction | ml | Reduction |
| P. auruginosa | 1.8 × 10⁵ | 2.0 | 99.998% | <1.0 | >99.9998% | <1.0 | >99.9998% | <1.0 | >99.9998% | <1.0 | >99.9998% |
| ATCC# 15442 | | | | | | | | | | | |
| K. pneumonia | 3.4 × 10⁵ | <1.0 | >99.9998% | <1.0 | >99.9998% | <1.0 | >99.9998% | <1.0 | >99.9998% | <1.0 | >99.9998% |
| ATCC# | | | | | | | | | | | |
| BAA1705 | | | | | | | | | | | |
| VRE | 4.3 × 10⁶ | <1.0 | >99.9998% | <1.0 | >99.9998% | <1.0 | >99.9998% | <1.0 | >99.9998% | <1.0 | >99.9998% |
| ATCC# | | | | | | | | | | | |
| 700221 | | | | | | | | | | | |

*Initials were performed by adding 0.1 mL of the selected bacteria to 100 ml of DI then removing a sample at 0 minutes, 90 minutes, and 150 minutes in the same manner as the treated samples. This solution was then diluted in Phosphate Buffer and plated in 0.1 and 1.0 ml duplicates. Neutralization controls were performed for each microorganism and plated in 0.1 ml and 1.0 mL at a 1/1000 dilution. Recoveries from neutralization controls, and positive controls did not vary significantly in the concentration of the recoverable bacteria.
**The calculation of each percent reduction at the indicated point is based upon average results from 0.1 ml and 1.0 ml volumes assayed in duplicates directly and at a 1/100 dilution.

TABLE 8
The recovery of indicated bacterial species at the indicated bacterial species at the indicated time points following exposure to the Tobrex

| | | Exposure Time | | | | | | | | | |
|---|---|---|---|---|---|---|---|---|---|---|---|
| | | 30 Minutes | | 60 Minutes | | 90 Minutes | | 120 Minutes | | 150 Minutes | |
| Micro- | | CFU/ | Percent** | CFU/ | Percent** | CFU/ | Percent** | CFU/ | Percent** | CFU/ | Percent** |
| organism | Initial* | ml | Reduction | ml | Reduction | ml | Reduction | ml | Reduction | ml | Reduction |
| P. | 1.8 × 10⁵ | 1.8 × 10² | 99.9% | 11 | 99.994% | 51 | 99.97% | <1.0 | >99.9994% | <1.0 | >99.9994% |
| auruginosa | | | | | | | | | | | |
| ATCC# | | | | | | | | | | | |
| 15442 | | | | | | | | | | | |
| K. | 3.4 × 10⁵ | 4.3 × 10³ | 98.7 | 1.9 × 10³ | 99.4% | 6.7 × 10² | 99.8% | 9.7 × 10² | 99.7% | 9.5 × 10² | 99.7% |
| pneumonia | | | | | | | | | | | |
| ATCC# | | | | | | | | | | | |
| BAA1705 | | | | | | | | | | | |
| VRE | 4.3 × 10⁶ | 2.8 × 10⁵ | 36.3% | 2.6 × 10⁵ | 39.4% | 2.2 × 10⁵ | 48.5% | 1.4 × 10⁵ | 36.6% | 6.6 × 10⁴ | 98.5% |
| ATCC# | | | | | | | | | | | |
| 700221 | | | | | | | | | | | |

*Initials were performed by adding 0.1 ml of the selected bacteria to 100 ml of DI then removing a sample at 0 minutes, 90 minutes, and 150 minutes in the same manner as the treated samples. This solution was then diluted in Phosphate Buffer and plated in 0.1 and 1.0 ml duplicates. Neutralization controls were performed for each microorganism and plated in 0.1 ml and 1.0 ml at a 1/1000 dilution. Recoveries from neutralization controls, and positive controls did not vary significantly in the concentration of the recoverable bacteria.
**The calculation of each percent reduction at the indicated point is based upon average results from 0.1 ml and 1.0 ml volumes assayed in duplicates directly and at a 1/100 dilution.

TABLE 9
The recovery of indicated bacterial species at the indicated time points following exposure to the Ciloxan

| | | Exposure Time | | | | | | | | | |
|---|---|---|---|---|---|---|---|---|---|---|---|
| | | 30 Minutes | | 60 Minutes | | 90 Minutes | | 120 Minutes | | 150 Minutes | |
| Micro- | | CFU/ | Percent** | CFU/ | Percent** | CFU/ | Percent** | CFU/ | Percent** | CFU/ | Percent** |
| organism | Initial* | mL | Reduction | mL | Reduction | mL | Reduction | mL | Reduction | mL | Reduction |
| P. auruginosa | 1.8 × 10⁵ | 8.1 × 10⁴ | 54.7% | 6.1 × 10⁴ | 65.7% | 6.3 × 10⁴ | 64.4% | 5.0 × 10⁴ | 72.1% | 4.1 × 10⁴ | 77.2% |
| ATCC# 15442 | | | | | | | | | | | |
| K. pneumonia | 3.4 × 10⁵ | 9.7 × 10⁴ | 71.1% | 1.0 × 10⁵ | 70.0% | 9.8 × 10⁴ | 71% | 2.2 × 10⁵ | 50% | 7.4 10⁴ | 78% |
| ATCC# | | | | | | | | | | | |
| BAA1705 | | | | | | | | | | | |
| VRE | 4.3 × 10⁶ | 3.1 × 10⁵ | 28.7% | 2.2 × 10⁵ | 49.6% | 2.3 × 10⁵ | 46.5% | 7.6 × 10⁴ | 77.6% | 1.7 × 10⁵ | 60.4% |
| ATCC # 700221 | | | | | | | | | | | |

*Initials were performed by adding 0.1 mL of the selected bacteria to 100 mL of DI then removing a sample at 0 minutes, 90 minutes, and 150 minutes in the same manner as the treated samples. This solution was then diluted in Phosphate Buffer and plated in 0.1 and 1.0 mL duplicates. Neutralization controls were performed for each microorganism and plated in 0.1 mL and 1.0 mL at a 1/1000 dilution. Recoveries from neutralization controls, and positive controls did not vary significantly in the concentration of the recoverable bacteria.
**The calculation of each percent reduction at the indicated point is based upon average results from 0.1 mL and 1.0 mL volumes assayed in duplicates directly and at a 1/100 dilution.

The composition 127-35B which was highly irritating to the eye was modified principally by removing the sodium lauryl sulfate which is well known to be irritating to nostrils and epithelial tissue. The composition 127-35B contained 2.4 mg of silver ion (Ag⁺) per gram of material. The composition 127-39T contains 2.5 mg of silver ion (Ag⁺) per ml of the composition. Consequently, it is not surprising that the activity of these two preparations in inhibiting bacteria is essentially nearly identical (Compare Table 1 to Table 7 for their inhibition of the microorganisms at various times).

Both in the experiments with the compositions 127-35B and 127-39T the organisms utilized were subjected to the antimicrobial agents at 100 times the dilution of these agents so that, for example, in working with 127-35B and 127-39T the actual concentrations that the organisms were subjected to is 0.024 mg and 0.025 for 127-35B and 127-39T, respectively.

Although Tobramycin (Table 8) showed superior antimicrobial activity for the three organisms tested as compared to Ciloxan (Table9) the Vancomycin-resistant organism required at least 150 minutes of exposure to the Tobramycin before showing a per cent reduction of bacteria of 98.5% and Ciloxan never was able to achieve more than 60.4% after 150 minutes for the Vancomycin-resistant organism (Table 9).

In addition to the significant advantages of the silver ion compositions in suppressing a multitude of microorganisms, the cost of the silver ion preparations would be significantly less than the antibiotics tested. For example, the ophthalmic ointment of Ciloxan in a 3.5 gram tube at a concentration of Ciloxan of 0.3% has a retail price of $171.99. A 3.5 gram quantity of the Tobrex ophthalmic ointment at a concentration of 0.3% has a retail price of $131.99.

### Effect of Preparation 127-39T on Viruses

Because ophthalmological infections may also be caused by viruses, we felt it significant to test at least two viruses against the preparation 127-39T.

An aliquot of Poliovirus (ATCC VR-1562, Chat Lsc 1) was added to solution (1 gram 127-39T pre- suspended in 100 mL of DI water). Samples were taken at specified time periods (30, 60, and 120 minutes) and combined with an equal amount of 2× DE Neutralizing Broth (Neogen). For enumeration BGM cell monolayers were inoculated with diluted aliquots of the samples. Following infection, the cells were overlaid with media and incubated for 48-72 hours at 36.5 degrees C. at 5% CO2. The resulting plaques on the monolayers were counted and the plaque forming units per milliliter (PFU/ml) for each sample was calculated based on the average of at least 5 replicates.

TABLE 10
Efficacy of 127-39T formulation against Poliovirus
performed on Nov. 30, 2012

| 0 Minutes | 30 Minutes | 30 Minute | 60 Minutes | 60 Minute |
|---|---|---|---|---|
| poliovirus | Poliovirus | percent | Poliovirus | percent |
| pfu/ml | Pfu/ml | Reduction | Pfu/ml | reduction |
| 118000 | 12200 | 89.66% | 6800 | 94.24% |

TABLE 11
Efficacy of 127-39T against the coliphage MS2 performed on Nov. 28, 2012

| | | 3 Minute | | 5 Minute | | 15 Minute |
|---|---|---|---|---|---|---|
| 0 Minutes | 3 Minutes | Percent | 5 Minutes | Percent | 15 Minutes | Percent |
| MS2 pfu/ml | MS2 pfu/ml | Reduction | MS2 pfu/ml | Reduction | MS2 pfu/ml | Reduction |
| 220000 | 121700 | 44.7% | 115400 | 47.5% | 20400 | 90.7% |

| | | | 60 Minute |
|---|---|---|---|
| 30 Minutes | | 60 Minutes | Percent |
| MS2 pfu/ml | MS2 pfu/ml | MS2 pfu/ml | Reduction |
| 5750 | 97.4% | 1700 | 99.2% |

An aliquot of MS2 (ATCC -B1) coliphage was added to the solution (1 gram 127-39T in 100 ml of DI water). Samples were then assayed using a double agar overlay plaques assay using E. coli C3000 directly and at 1100 in volumes of 0.1 ml and 1.0 ml. The results are based upon the averages read on Nov. 20, 2012.

The above descriptions and examples illustrate particular constructions including the preferred embodiments of the solutions. However, the invention is not limited to the precise constructions described herein, but, rather, all modifications and improvements thereof encompassed within the scope of the invention.

Many of the examples described herein utilize the surface active agents such as those characterized as Aerosal OT. These surface-active agents are utilized primarily to effect a homogenous dispersion between the non-aqueous soluble components with the aqueous soluble components in order to ensure homogeneity.

These surface active agents are also utilized in order to improve wetting of a medical dressing or bandage in the event that a wound may be exudating, and the enhanced wicking in such a bandage or medical dressing serves to quickly absorb any blood or serum from a wound site into the dressing. Other surface active agents, such as a member of the group of Tweens: (Tween 20, polyoxyethylene sorbitan monolaurate; Tween 40, polyoxyethylene sorbitan monopalmitate; or Tween 85, polyoxyethylene sorbitan trioleate may be incorporated into the composition without deviating from the novelty of the invention described herein.

The preferred white mineral oil and white petrolatum used to enhance the viscosity of the composition 127-39T may be modified by those skilled in the art with regard to the purity of these ingredients and their concentration without deviating from the novelty of the invention described herein.is

Although the aqueous soluble silver salt preferred as a preferred embodiment herein is silver nitrate, other aqueous soluble silver salts may be utilized by those skilled in the art without deviating from the novelty of the invention described herein.

### REFERENCES

Alcon, Inc. (ophthalmological products) headquarters in Hünenberg, Switzerland and Fort Worth, Tex.

Argyrol Pharmaceuticals Corporation, 1715 Galatea Terrace, Corona Del Mar, Calif.

Azary Technologies LLC 510(k) K040518

Fishman, Marshall L. and Joseph J. Jen—Editors—Chemistry and Function of Pectins, 1986.

Goodman, L. and A. Gilman (1965 The Pharmacological Basis of Therapeutics, 3^{rd} Ed. New York, the Macmillan Company

Illingworth, et al. (J. Heart Valve Dis. 1998 Septmeber; 7(5): 524-30)

McDowell, R. H.—Properties of Alginates, Alginate Industries Limited, Second Edition, 1961 Bedford Street Strand, London England

Meaders, Brian C. and John M. Azar “Bacterial Conjunctivitis: a review of therapies and approaches” ADVANCE for NPs & PAs 3 (11):—Page 25-9, 34—November 2012

Nussinovitch, A.—Hydrocolloid Application—Published by Springer US-1997

The Merck Index, 7^{th} Edition. Stecher, P. G., Ed. Rahway, N.J. Merck & Co., Inc., 1960

Spectrum Laboratory Products, Inc. Gardena, Calif.—MSDS Sheet Sep. 13, 2006

Süpfle, K. and Werner, R. (1951 Microdeosimetric investigation of the olygodynamic effect of silver. Mikrochemie ver. Mikrochim. Acta., 36/37, 866-881

The United States Pharmacopeia of America—Eighteenth Revision, Sep. 1, 1970, p. 02-103

Trop, Marija, et al. Silver coated Dressing Acticoat Caused Raised Liver Enzymes and Argyria-like Symptoms in Burn Patient. The Journal of TRAUMA Injury, Infection, and Critical Care Volume 60 Number 3:648-652, March 2006.

### U.S. Patents

| Pat. No. | Issue Date | Inventor |
|---|---|---|
| 5,674,524 | Oct. 7, 1997 | Scherr |
| 5,718,916 | Feb. 17, 1998 | Scherr |
| 7,128,929 | Oct. 31, 2006 | Scherr |
| 6,696,077 | Feb. 24, 2004 | Scherr |
| 5,147,648 | Sep. 15, 1992 | Bannert |
| 5,688,923 | Nov. 18, 1997, | Gerrish |
| 3,639,575 | Feb. 1, 1972 | Schmolka |
| 4,148,974 | Jan. 22, 1980 | Van Leuven |

### Foreign Patents

| Pat. No. | Issue Date | Inventor |
|---|---|---|
| 221859 (India) | Jul. 8, 2008 | Scherr |
| GB 2,357,765 (Great Britain) | Apr. 21, 2004 | Scherr |
| 2322266 (Russia) | Feb. 18, 2003 | Scherr |

1. The process for making a cation cross-linked pectin composition comprising:
a) Cross-linking the pectin with an aqueous soluble silver salt in distilled or deionized water and,
b) with continuous stirring, adding to the silver pectate suspension, a non-pectin plasticizing agent and,
c) a surface active agent.

2. The process of claim 1 wherein the non-pectin plasticizing agent is selected from the group, consisting of cellulose, carboxymethylcellulose, carboxymethyl ethyl cellulose, hyaluronic acid, carrageenan, and/or gellan gum.

3. The process of claim 2 wherein the non-pectin plasticizing agent is cellulose.

4. The process of claim 1 where the aqueous soluble silver salt may be silver acetate, silver fluogallate, silver nitrate, and/or silver sulfate.

5. The process of claim 4 wherein the aqueous soluble silver salt is silver nitrate.

6. The process of claim 1 wherein the upper limit of the DE of the pectin that is cross-linked with the aqueous soluble silver salt is 50%.

7. The process of claim 1 wherein the upper limit of the DE of the pectin that is cross-linked with the aqueous soluble silver salt is 30%.

8. The process of claim 1 wherein the upper limit of the DE of the pectin that is cross-linked with the aqueous soluble silver salt is 5%.

9. The process of claim 1 wherein the lower limit of the DE of the pectin that is cross-linked with the aqueous soluble silver salt is 0%.

10. The process of claim 1 wherein a surface active agent may be selected from the group ammonium lauryl sulfate, Tween 80 and/or Tween 20.

11. The process of claim 10, wherein Tween 80 is added to the silver pectate composition.

12. The process of claim 1 wherein the upper limit of the DA of the pectin that is cross-linked with the aqueous soluble silver salt is 25%.

13. The process of claim 1 wherein the upper limit of the DA of the pectin that is cross-linked with the aqueous soluble silver salt is 20%.

14. The process of claim 1 wherein the lower limit of the DA of the pectin that is cross-linked with the aqueous soluble silver salt is 0%.

15. The process of claim 1 wherein the lower limit of the DA of the pectin that is cross-linked with the aqueous soluble silver salt is 5%.

16. The process of claim 1 wherein the lower limit of the DA of the pectin that is cross-linked with the aqueous soluble silver salt is 10%.

17. The process for making a cation cross-linked pectin composition comprising:
a) Cross-linking the pectin with an aqueous soluble silver salt in distilled or deionized water and,
b) with continuous stirring, adding to the silver pectate suspension, a non-pectin plasticizing agent,
c) a surface active agent and,
d) a medicament.

18. The process of claim 17 wherein the medicament may be an antimicrobial agent, a sugar, a preservative, an antibody, and/or an isotope.

19. The process of claim 18 wherein the sugar may be a lactose and/or dextrose.

20. The process of claim 17 wherein the aqueous soluble silver salt may be silver acetate, silver fluogallate, silver nitrate, and/or silver sulfate.

21. The process of claim 21 wherein the aqueous soluble silver salt is silver nitrate.

22. The process of claim 18 wherein the preservative may be a mercury compound, a silver colloid, a phenolic compound, and/or chromium.

23. The process of claim 18 wherein the antibody may be antimicrobial.

24. The process of claim 18 wherein the isotope may be boron.

25. The process for making a cation cross-linked pectin composition comprising:
a) Cross-linking the pectin with an aqueous soluble silver salt is distilled or deionized water and,
b) with continuous stirring, adding to the silver pectate suspension, a non-pectin plasticizing agent,
c) a surface active agent,
d) petrolatum and,
e) mineral oil.

26. The process of claim 25 wherein the non-pectin plasticizing agent is selected from the group, consisting of cellulose, carboxymethylcellulose, carboxymethyl ethyl cellulose, hyaluronic acid, carrageenan, and/or gellan gum.

27. The process of claim 25 wherein the non-pectin plasticizing agent is cellulose.

28. The process of claim 25 where the aqueous soluble silver salt may be silver acetate, silver fluogallate, silver nitrate, and/or silver sulfate.

29. The process of claim 25 wherein the aqueous soluble silver salt is silver nitrate.

30. The process of claim 25 wherein the petrolatum is white petrolatum.

31. The process of claim 25 wherein the mineral oil is white mineral oil.

32. A silver pectate moiety.
